(19) United States
(12) Patent Application Publication
(10) Pub. No.: US 2009/0156793 A1
(43) Pub. Date: Jun. 18, 2009

(54) Circular Chromosomes

(76) Inventors: Larry Gilbertson, Chesterfield, MO (US); Jason Ward, St. Charles, MO (US)

Correspondence Address: MONSANTO COMPANY, 800 N. LINDBERGH BLVD., ATTENTION: GAIL P. WUELLNER, IP PARALEGAL, (E2NA), ST. LOUIS, MO, 63167, US

(21) Appl. No.: 12/333,571

(22) Filed: Dec. 12, 2008

Related U.S. Application Data
(60) Provisional application No. 61/013,023, filed on Dec. 12, 2007.

(51) Int. Cl. C07H 21/04

(52) U.S. Cl. 536/23.1

(57) ABSTRACT

Circular chromosomes with centromere DNA comprise recombination sites flanking a region with bacterial replication DNA and a recombinase transcription unit. Circular chromosomes without bacterial replication DNA are formed by removing bacterial replication DNA from a circular chromosome by action of a recombinase that excises DNA between said recombination sites.

### CROSS REFERENCE TO RELATED APPLICATION

This application claims the benefit under 35 U.S.C. 119(e) of provisional application Ser. No. 61/013,179, filed Dec. 12, 2007, the disclosure of which is incorporated herein by reference in its entirety.

### FIELD OF THE INVENTION

Disclosed herein are circular chromosomes and methods of making and using circular chromosomes, including methods for removing DNA from circular chromosomes.

### BACKGROUND

Small circular artificial chromosomes as disclosed by Birchler et al. in WO 07134122 and by Copenhaver et al. in U.S. Pat. No. 7,193,128 are useful in providing a plurality of heterologous genes in transgenic organisms such as plants. In the production of such artificial chromosomes it is useful to incorporate bacterial replication DNA that allows replication of the chromosome in bacteria.

### SUMMARY OF THE INVENTION

This invention provides methods of removing DNA from circular chromosomes comprising a centromere DNA region and a bacterial replication DNA region. In aspects of the invention the bacterial replication DNA is flanked by recombination sites or meganuclease sites. Such methods comprise providing in a plant cell containing a circular chromosome a recombinase transcription unit comprising a promoter active in plant cells operably linked to DNA for expressing a recombinase that will excise from the circular chromosome bacterial replication DNA that is flanked by recombination sites that are recognized by the recombinase. Such methods also comprise providing in a plant cell containing a circular chromosome a meganuclease transcription unit comprising a promoter active in plant cells operably linked to DNA for expressing a meganuclease that will excise from the circular chromosome bacterial replication DNA that is flanked by sites that are recognized by the meganuclease. The methods can be practiced by providing such a recombinase transcription unit or meganuclease unit on the circular chromosome containing the bacterial replication DNA to be removed. The methods can also be practiced by proving such a recombinase transcription unit or meganuclease unit in the cell separately from the circular chromosome, e.g., on a separate plasmid or DNA fragment. The promoter that is active in plant cells that is in the recombinase transcription unit or the meganuclease transcription unit can be a constitutive or non-constitutive promoter, e.g., a tissue specific promoter or an inducible promoter.

In one aspect the invention also provides a method for removing marker DNA from a circular chromosome, e.g., one or more marker transcription units that are flanked by recombination or meganuclease sites. For example, when a common recombinase is used to remove bacterial replication DNA and marker DNA, it is useful to use a non-constitutive promoter to express the recombinase, e.g., allowing a selectable marker protein to be expressed for a time until a plant cell transformed with a circular chromosome has developed into a plant. Alternatively, separate recombinases or meganucleases or combinations of a recombinase and a meganuclease can be use to excise the bacterial replication DNA and the maker transcription units. Such a non-constitutive promoter for expressing the agent for removing the marker transcription unit can be inducible or tissue specific.

The methods of this invention can be practiced with any system of recombinase and recombination sites, e.g., a system where the recombination sites are LoxP sites and the recombinase is CRE recombinase or where the recombination sites are Frt sites and the recombinase is FLP recombinase.

In the various aspects of this invention it is generally desirable for the circular chromosome to further comprise transgenes, i.e., recombinant DNA that is transcribed as coding RNA (messenger RNA encoding a protein) or as non-coding RNA (e.g., RNA for suppressing expression of a gene) or as both coding and non-coding RNA. Optionally, the circular chromosome can comprise DNA insertion sites, telomeres, meganuclease recognitions sites or homing nuclease sites.

Another aspect of the invention provides circular chromosomes comprising a centromere DNA region and a bacterial replication DNA region flanked by recombination sites and wherein the circular chromosome further comprises a recombinase transcription unit comprising a promoter active in plant cells operably linked to DNA for expressing a recombinase that will excise DNA flanked by said recombination sites. Such a promoter active in plant cells for expressing a recombinase can be a constitutive or non-constitutive promoter. The circular chromosome can further comprises one or more marker transcription units that are flanked by recombination sites and that express a selectable or screenable marker protein in plant cells. A recombinase transcription unit for producing recombinase to excise marker transcription DNA desirably has a non-constitutive promoter that is active in plant cells, e.g., an inducible promoter or a tissue specific promoter.

A circular chromosome of this invention can further comprise between the centromere DNA region and a recombination site one or more RNA transcription units for expressing transcribed RNA in plant cells. A circular chromosome of this invention can further comprise a meganuclease transcription unit comprising a promoter active in plant cells operably linked to DNA for expressing a meganuclease; such a meganuclease transcription unit is flanked by meganuclease recognition sites so that, when the meganuclease is produced in plant cells, the DNA between the meganuclease recognition sites will be excised to linearize the circular chromosome. A circular chromosome of this invention can further comprise telomere regions adjacent to the meganuclease recognition sites so that a linearized chromosome will have telomeres. A circular chromosome of this invention can further comprise a homing nuclease site flanked by telomere regions to provide a linearized chromosome.

### BRIEF DESCRIPTION OF THE DRAWINGS

FIGS. 1-7 illustrate circular chromosomes.

### DETAILED DESCRIPTION OF THE INVENTION

Recombinant, circular chromosomes that are useful for practicing the aspects of this invention are disclosed by Copenhaver et al. in U.S. Pat. No. 7,193,128, which is incorporated herein by reference.

The replication of plasmids in host bacteria is well known in the art. The circular chromosome may comprise bacterial replication region DNA that provide replication function and antibiotic selection in host bacterial cells. For example, the chromosome may contain an E. coli origin of replication such as ori322, ori2, or a broad host range origin of replication such as oriV, oriRi or oriColE.

Methods of inserting recombinant chromosomes into plant cells are well known by persons of ordinary skill in the art. For instance, specific instructions for transforming plant cells by microprojectile bombardment with particles coated with recombinant DNA are found in U.S. Pat. No. 5,015,580 (soybean); U.S. Pat. No. 5,550,318 (corn); U.S. Pat. No. 5,538,880 (corn); U.S. Pat. No. 5,914,451 (soybean); U.S. Pat. No. 6,160,208 (corn); U.S. Pat. No. 6,399,861 (corn) and U.S. Pat. No. 6,153,812 (wheat); U.S. Pat. No. 6,002,070 (rice); U.S. Pat. No. 7,122,722 (cotton); U.S. Pat. No. 6,051,756 (Brassica); U.S. Pat. No. 6,297,056 (Brassica); and Patent Application Publication US 2004/0123342 A1 (sugarcane); and specific instructions for transforming plant cells by Agrobacterium-mediated transformation are described in U.S. Pat. No. 5,159,135 (cotton); U.S. Pat. No. 5,824,877 (soybean); U.S. Pat. No. 5,591,616 (corn); U.S. Pat. No. 6,384,301 (soybean); U.S. Pat. No. 5,750,871 (Brassica); U.S. Pat. No. 5,463,174 (Brassica) U.S. Pat. No. 5,188,958 (Brassica), all of which are incorporated herein by reference.

The circular chromosome may comprise a marker transcription unit that is active in plant cells for expressing a marker protein to identify cells that are transformed with the chromosome. The marker protein can be selectable, e.g., the marker transcription unit can have DNA that encodes a neomycin phosphotransferase conferring resistance to neomycin and kanamycin, DNA that encodes aminoglycoside adenyltransferase (aadA) conferring resistance to spectinomycin or streptomycin, or a gentamycin (Gm, Gent) or one of many other known selectable marker proteins. The marker protein can be screenable, e.g., the marker transcription unit can have DNA that encodes for 5-enolpyruvylshikimate-3-phosphate synthase (EPSPS) as described in U.S. Pat. No. 5,627,061 conferring resistance to glyphosate herbicide, or DNA that encodes bromoxynil nitrilase (Bxn) as described in U.S. Pat. No. 4,810,648 conferring resistance to Bromoxynil herbicide, or DNA that encodes for dicamba monooxygenase (DMO) as described in U.S. Pat. Nos. 7,105,724 and 7,022,896, and in US Patent Application Publication US2008/0015110 A1, conferring resistance to dicamba herbicide. The promoter in a marker transcription unit is typically a constitutive promoter that is active in plant cells, such as a nopaline synthase (NOS) promoter; the cauliflower mosaic virus (CaMV) 19S or 35S promoter as disclosed in U.S. Pat. No. 5,858,642; a figwort mosaic virus promoter (FMV) as disclosed in U.S. Pat. No. 6,051,753; or actin promoters, such as a rice actin promoter (Os.Act1) as disclosed in U.S. Pat. No. 5,641,876. When the use of a constitutive promoter is not desirable, a non-constitutive promoter such as an inducible or tissue specific promoter can be selected. Examples of useful inducible promoters include a cold inducible promoter as disclosed in U.S. Pat. No. 6,084,089, a light inducible promoter as disclosed in U.S. Pat. No. 6,294,714, and a salt inducible promoter as disclosed in U.S. Pat. No. 6,140,078. Examples of useful tissue specific promoters include a seed-specific promoter such as a napin promoter as disclosed in U.S. Pat. No. 5,420,034, a maize L3 oleosin promoter as disclosed in U.S. Pat. No. 6,433,252, a zein Z27 promoter or a glutelin 1 promoter as disclosed by Russell et al (1997) Transgenic Res., 6:157-166, a globulin 1 promoter as disclosed by Belanger et al. (1991) Genetics, 129:863-872, or a peroxiredoxin antioxidant promoter (Per1) as disclosed by Stacy et al. (1996) Plant Mol Biol., 31:1205-1216. There is normally a 3′ transcription termination region in a transcription unit which is operably linked to and located downstream of the coding region of a gene and which includes polynucleotides that provide polyadenylation signal and other regulatory signals capable of affecting transcription, mRNA processing or gene expression. The polyadenylation sequence can be derived from a variety of plant genes or from T-DNA genes. Commonly used 3′ transcription termination region are derived from Pisum sativum RbcS2 gene (Coruzzi et al. (1984), EMBO J, 3: 1671) and Agrobacterium tumefaciens nopaline synthase gene (GenBank Accession E01312).

Recombination systems which are useful in excising DNA from circular chromosomes include the CRE-lox system from bacteriophage P1 as disclosed in U.S. Pat. No. 4,959,317; the FLP-frt system from yeast as disclosed in U.S. Pat. No. 5,527,695, the Gin/gix system from phage Mu (Maeser et al, Mol. Gen. Genet., 230(1-2):170-176, 1991) and the R/RS system of the pSR1 plasmid from Xygosaccharomyces rouxii (Onouchi et al., Nuc. Acids Res., 19:6373-6378, 1991.

Meganucleases and homing endonucleases and their use are known in the art. See, for example, U.S. Pat. Nos. 5,792,632; 5,830,729; 6,566,579; 7,098,031; 7,285,538; and 7,338,800 and in Patent Application Publications US 2002/0107214 A1, US 2002/0187508 A1, US 2003/0106077 A1, US 2003/0182670 A1, US 2003/0224481 A1, US 2004/0002092 A1, US 2004/0019002 A1, US 2004/0126883 A1, US 2005/0032223 A1, US 2005/0059022 A1, US 2005/0090010 A1, US 2005/0120395 A1, US 2005/0172365 A1, US 2006/0078552 A1, US 2006/0153826 A1, US 2006/0253916 A1, US 2007/0014769 A1, US 2007/0117128 A1, US 2007/0134796 A1, US 2007/0141038 A1, US 2008/0050819 A1, US 2008/0134351 A1, US 2008/0241915 A1, US 2008/0271166 A1 and US 2006/0206949 A1., the disclosures of which relating to meganucleases and homing nucleases and their use for the methods of this invention are incorporated herein by reference.

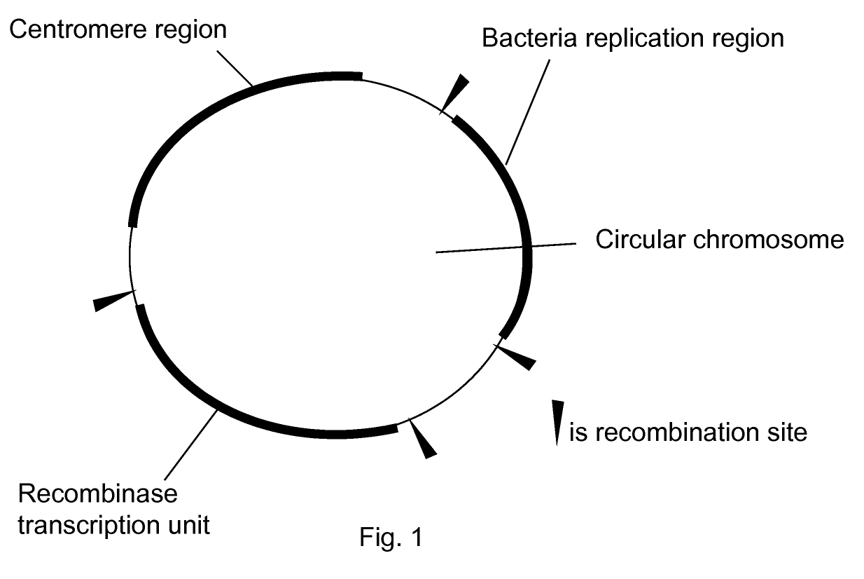

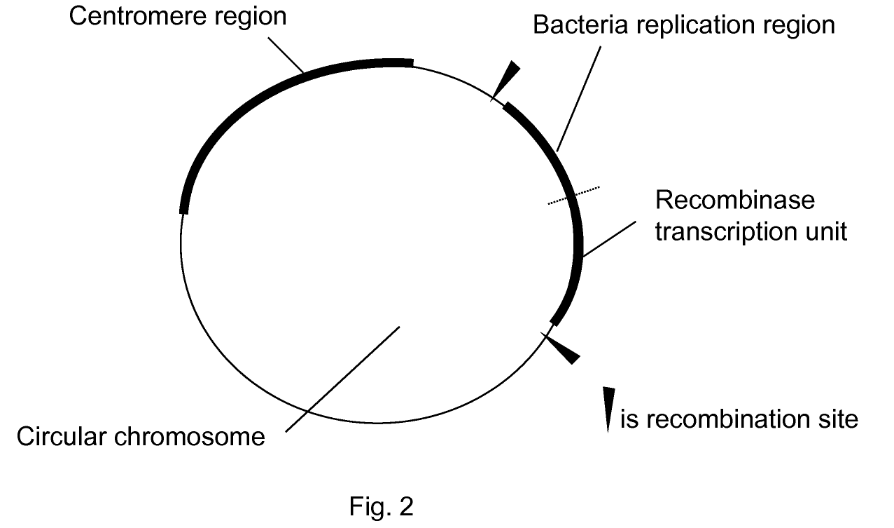

With reference to FIG. 1 there is shown a circular chromosome having a plant centromere DNA region and a bacterial replication DNA region flanked by recombination sites, e.g., as disclosed by Copenhaver et al. in U.S. Pat. No. 7,193,128. The circular chromosome as provided by this invention further has a recombinase transcription unit DNA flanked by recombination sites. When the recombinase transcription unit comprises a promoter that is active in plant cells operably linked to DNA encoding a CRE recombinase, the recombinase will be expressed when the chromosome is located in a plant cell to effect excision of the DNA segments flanked by Lox recombination sties, e.g., LoxP sites, to provide a circular chromosome without bacterial replication region DNA and without recombinase transcription unit DNA. The bacterial replication region DNA and the recombinase transcription unit DNA can be separated as shown in FIG. 1 or can be contiguous and flanked by a single pair of recombination sites as illustrated in FIG. 2.

In another aspect of the invention a circular chromosome has a plant centromere DNA region and a bacterial replication DNA region which is flanked by recombination sites. Such a circular chromosome is co-transformed into a plant cell with either a plasmid containing a recombinase transcription unit or a DNA fragment containing a recombinase transcription unit. Recombinase can be produced from such a plasmid or DNA fragment whether or not the plasmid or fragment becomes stably integrated into the host genome. When such a plasmid or DNA fragment is stably integrated into a native chromosome unlinked from the artificial chromosome, the recombinase transcription unit can be segregated away from the circular chromosome.

In another aspect of the invention the recombinase transcription unit is stably integrated into the genome of a plant which can be crossed with a plant of the same species which has been transformed with a circular chromosome carrying a bacterial replication DNA flanked with recombination sites. After the DNA flanked by the recombination sites has been excised, the recombinase transcription unit is segregated away by outcrossing.

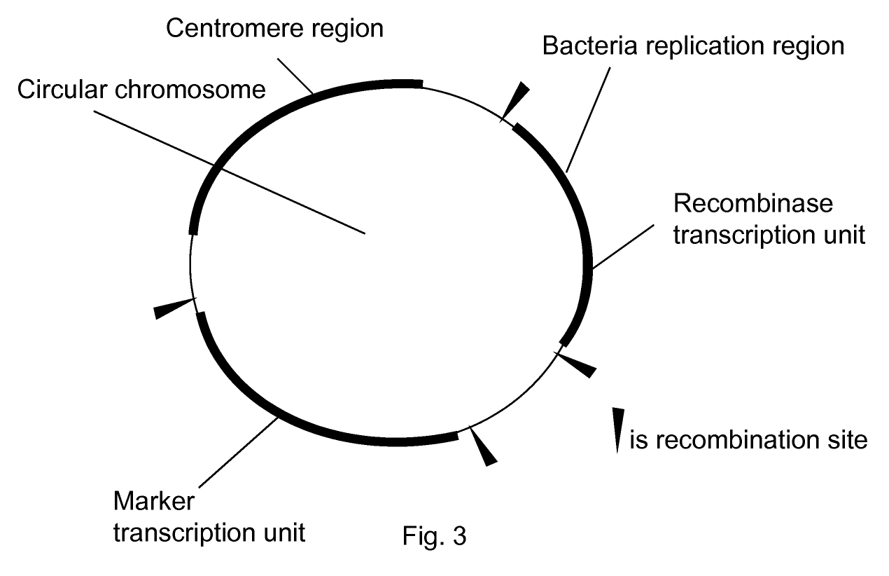

With reference to FIG. 3 there is shown a circular chromosome further having a marker transcription unit DNA region comprising a promoter that is active in plant cells and is operably linked to DNA encoding a selectable or screenable marker protein. Because the marker transcription unit is required to be actively expressing a marker for sufficient time to isolate and recover a cell transformed with the circular chromosome, the use of a constitutive promoter that is active in plant cells may not be preferred for use in the recombinase transcription unit. Rather, it may be advantageous that the recombinase transcription unit comprise a promoter that is inducible or tissue specific.

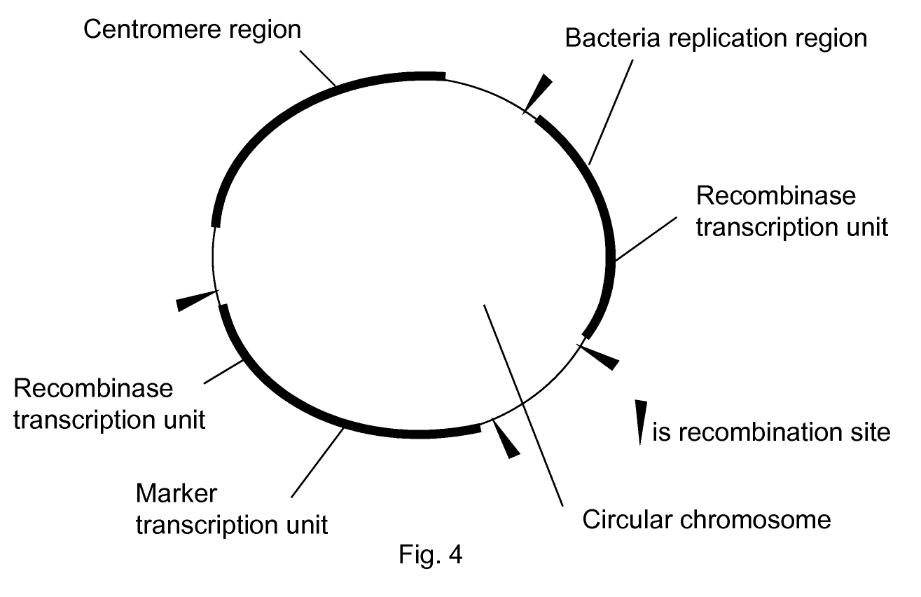

With reference to FIG. 4, there is shown a circular chromosome having two distinct recombinase transcription units that can be active at different times. The recombinase transcription unit adjacent to the bacterial replication region can be active as soon as the chromosome is inserted into a plant cell to quickly excise the bacterial replication region and adjacent recombinase transcription unit. The recombinase transcription unit adjacent to the marker transcription unit is preferably active after the cell transformed with the recombinant chromosome is identified and isolated, e. g., as late as during the production of progeny cells.

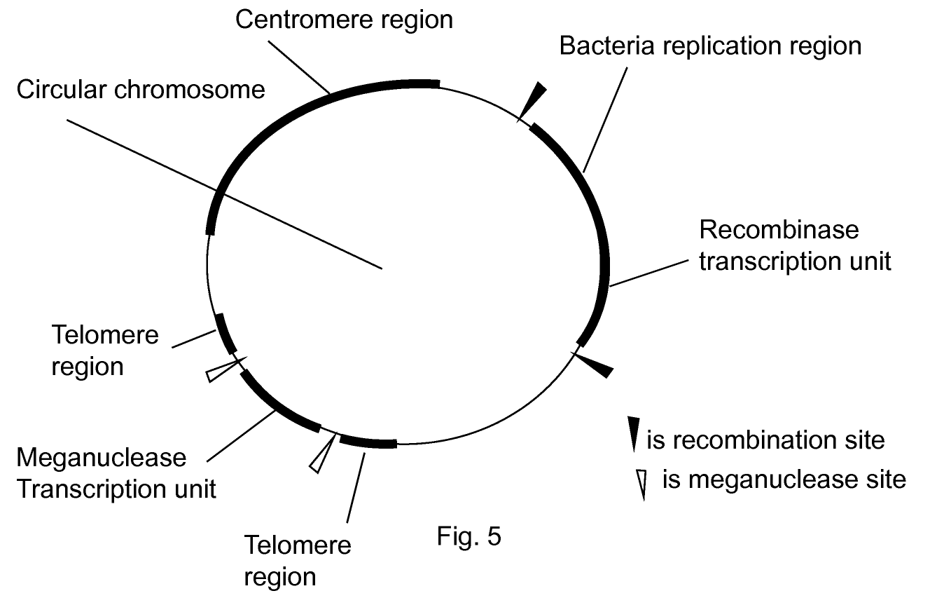

With reference to FIG. 5 there is shown a circular chromosome that is amenable to linearization. The chromosome has a bacterial replication region and adjacent recombinase transcription unit flanked by recombination sites to allow for excision of the DNA between the recombination sites. The chromosome also has two regions of telomere DNA flanking a pair of meganuclease sites and a meganuclease transcription unit. The meganuclease transcription unit comprises a promoter that is active in plant cells operably linked to DNA encoding a meganuclease that excises the DNA between the meganuclease sites to linearize the circular chromosome providing a linear chromosome with terminal telomere DNA. Such a circular chromosome would also comprise a marker transcription unit which is not shown.

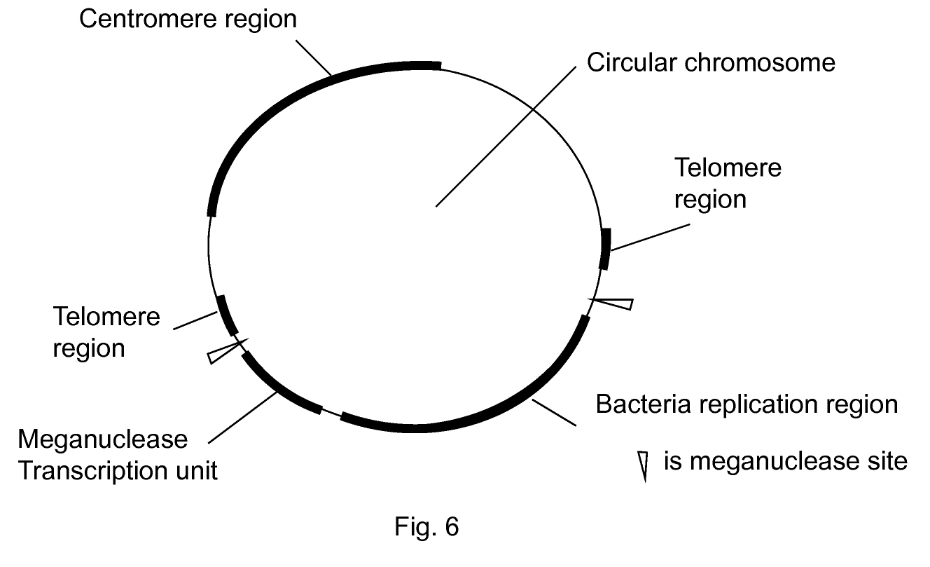

With reference to FIG. 6 there is shown a circular chromosome that is amenable to linearization. The chromosome comprises a bacterial replication region adjacent to a meganuclease transcription unit flanked by meganuclease transcription sites adjacent to regions of telomere DNA. Expression of the meganuclease by a promoter that is active in plant cells effects excision of the bacterial replication region and the adjacent meganuclease transcription unit to linearize the circular chromosome providing a linear chromosome with terminal telomere DNA. Such a circular chromosome would also comprise a marker transcription unit which is not shown.

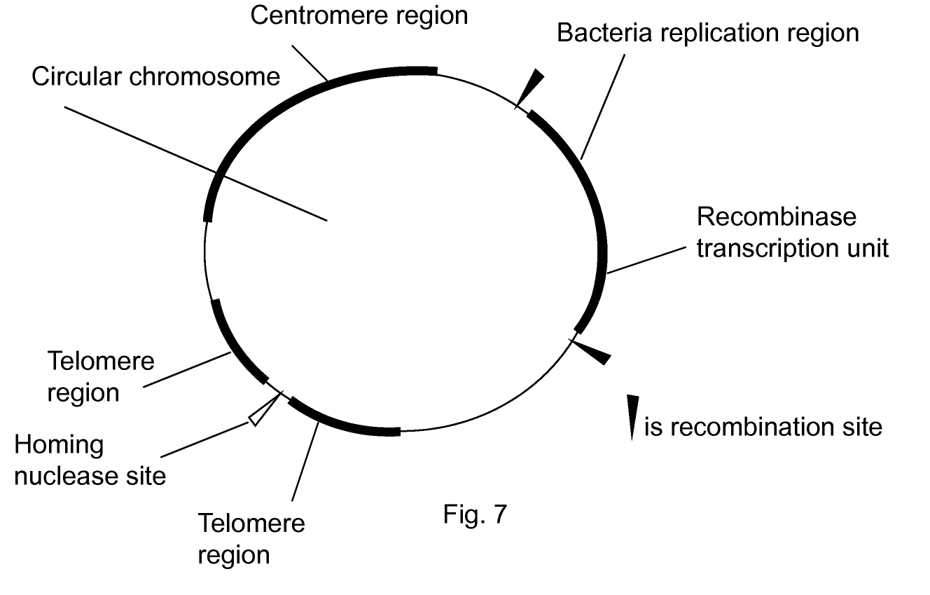

With reference to FIG. 7 there is shown a circular chromosome that is amenable to linearization. The chromosome has a bacterial replication region and adjacent recombinase transcription unit flanked by recombination sites to allow for excision of the DNA between the recombination sites. The chromosome has two regions of telomere DNA flanking a homing nuclease site. Expression of a homing nuclease in a plant cell transformed with the circular chromosome will effect linearization of the circular chromosome. Such a circular chromosome would also comprise a marker transcription unit which is not shown.

In another aspect of the invention the circular chromosome further comprises between said centromere DNA region and a recombination site one or more transgenes comprising recombinant DNA that is transcribed as coding RNA (messenger RNA encoding a protein) or as non-coding RNA (e.g., RNA for suppressing expression of a gene) or as both coding and non-coding RNA. DNA's for transgenes are illustrated in PCT/US2007/080323 published as WO08063755A2. Combinations of multiple transgenes are disclosed in US 2008/0256669 A1.

### EXAMPLE 1

This example illustrates removal of DNA from a circular chromosome. With reference to FIG. 2 a circular chromosome was prepared with LoxP sites flanking a bacterial replication DNA region comprising a CRE recombinase transcription unit comprising a constitutive promoter functional in plant cells, i.e., a CaMV35S promoter, operably linked to the DNA encoding CRE recombinase. The circular chromosome was then transformed into corn embryos by microparticle bombardment to produce 122 events of transgenic cells containing circular chromosomes which were grown into transgenic plants. Cells from plants from the events were tested for the presence/absence of the bacterial replication DNA region and the CRE recombinase transcription unit by TaqMan® assay. For transgenic plant cells from about 68% of the events the assay tested negative for the presence of the bacterial replication DNA region and the CRE transcription unit. Transgenic plant cells for a subset of those 68% of the events were assayed as positive for the presence of a circular chromosome which indicated transgenic plants having cells with a circular chromosome DNA as being autonomous from a native chromosome DNA by fluorescence in situ hybridization (FISH). The FISH assay is disclosed by Carlson et al., (2007) PLoS Genetics, 3: e179.

### EXAMPLE 2

This example illustrates an alternate method of removing DNA from a circular chromosome. Four separate circular chromosomes each containing a different sized fragment of maize centromere ranging from about 20 to 100 kilobases and containing a bacterial replication DNA region flanked by LoxP sites were paired with a plasmid comprising a CRE recombinase transcription unit that is expressed in plants. The pairs of circular chromosomes and CRE recombinase plasmids were transformed into corn tissue by microparticle bombardment producing multiple transgenic events of transgenic plants. Cells from plants for 249 transgenic events were assayed for the presence of the CRE recombinase transcription unit and the bacterial replication DNA region by TaqMan assay. Cells from some of the events contained the circular chromosome without the bacterial replication DNA but with the CRE recombinase transcription unit stably integrated into the genome. Cells from about 51% of the events contained the circular chromosome without the bacterial replication DNA and without the CRE recombinase transcription unit.

### EXAMPLE 3

This example illustrates an alternate method of removing DNA from a circular chromosome. With reference to FIG. 6 a circular chromosome is prepared with maize centromere and telomere DNA regions and meganuclease recognition sites flanking a region containing a bacterial replication DNA region and a meganuclease transcription unit. The meganuclease transcription unit comprises a promoter functional in plant cells operably linked to DNA encoding a meganuclease. The circular chromosome is transformed into corn tissue by microparticle bombardment producing transgenic events which are assayed for the presence of the bacterial replication DNA region by TaqMan® assay. Plant cells from some of the events contained the circular chromosome without the bacterial replication DNA.

What is claimed is:

1. A method of removing DNA from a circular chromosome in a plant cell wherein said circular chromosome comprises a plant centromere DNA region and further comprises a bacterial replication DNA region that is flanked by recombination sites, said method comprising providing in said plant cell a recombinase transcription unit comprising a promoter that is functional in said plant cell and is operably linked to DNA encoding a recombinase that will excise DNA between said recombination sites whereby, when said recombinase transcription unit is expressed in said plant cell to produce said recombinase, said bacterial replication DNA region is removed from said circular chromosome.

2. A method of claim 2, wherein said recombinase transcription unit is located on said circular chromosome.

3. The method of claim 1, wherein said recombinase transcription unit is provided in said plant cell on a plasmid or DNA fragment separate from said circular chromosome.

4. The method of claim 1, wherein said circular chromosome further comprises a marker transcription unit that comprises a promoter that is active in plant cells operably linked to DNA encoding a selectable or screenable marker protein and wherein said circular chromosome comprises sites that allow said marker transcription unit to be removed by action of a recombinase or meganuclease.

5. The method of claim 4, wherein said the promoter in the recombinase transcription units is a non-constitutive promoter that is inducible or tissue specific.

6. The method of claim 1, wherein said circular chromosome further comprises between said centromere DNA region and a recombination site one or more transgenes for expressing transcribed RNA in plant cells.

7. The method of claims 1, wherein said recombination sites are LoxP sites and said recombinase is CRE recombinase, or wherein said recombination sites are Frt sites and said recombinase is FLP recombinases.

8. A circular chromosome comprising a centromere DNA region and a bacterial replication DNA region flanked by recombination sites and wherein said circular chromosome further comprises a recombinase transcription unit comprising a promoter that is functional in said plant cell and is operably linked to DNA encoding a recombinase that will excise DNA flanked by said recombination sites.

9. The circular chromosome of claim 8, wherein said promoter that is functional in said plant cell and is operably linked to DNA encoding a recombinase is a constitutive or non-constitutive promoter.

10. The circular chromosome of claim 8, wherein said circular chromosome further comprises a marker transcription unit that comprises a promoter that is active in plant cells operably linked to DNA encoding a selectable or screenable marker protein and wherein said circular chromosome comprises sites that allow said marker transcription unit to be removed by action of a recombinase or meganuclease.

11. The circular chromosome of claim 10, wherein said promoter that is in said recombinase transcription unit is a non-constitutive promoter.

12. The circular chromosome of claim 8, that further comprises between said centromere DNA region and a recombination site one or more RNA transcription units for expressing transcribed RNA in plant cells.

13. The circular chromosome of claim 8, wherein said recombination sites are LoxP sites and said recombinase is CRE recombinase, or wherein said recombination sites are Frt sites and said recombinase is FLP recombinase.

14. The circular chromosome of claim 8, further comprising a meganuclease transcription unit comprising a promoter active in plant cells operably linked to DNA for expressing a meganuclease wherein said meganuclease transcription unit is flanked by meganuclease recognition sites, whereby, when said meganuclease is produced in plant cells, the DNA between said meganuclease recognition sites will be excised to linearize said circular chromosome.

15. The circular chromosome of claim 14, further comprising telomere regions adjacent to said meganuclease recognition sites.

16. The circular chromosome of claim 8, further comprising a homing nuclease site flanked by telomere regions.

17. A circular chromosome comprising a centromere DNA region and a bacterial replication DNA region flanked by meganuclease sites, wherein said circular chromosome further comprises a meganuclease transcription unit comprising a promoter active in plant cells operably linked to DNA for expressing a meganuclease whereby, when said circular chromosome is inserted into a plant cell, the meganuclease will be produced and effect excision of the DNA between said meganuclease recognition sites to linearize said circular chromosome.
